(19) United States
(12) Patent Application Publication
(10) Pub. No.: US 2025/0087355 A1
(43) Pub. Date: Mar. 13, 2025

(54) A MACHINE LEARNING BASED FRAMEWORK USING ELECTRORETINOGRAPHY FOR DETECTING EARLY STAGE GLAUCOMA

(71) Applicants: Peter Koulen, Leawood, KS (US); Amirfarhang Mehdizadeh, Shawnee, KS (US); Mohan Kumar Gajendran, Kansas City, MO (US)

(72) Inventors: Peter Koulen, Leawood, KS (US); Amirfarhang Mehdizadeh, Shawnee, KS (US); Mohan Kumar Gajendran, Kansas City, MO (US)

(21) Appl. No.: 18/720,485

(22) PCT Filed: Dec. 16, 2022
(86) PCT No.: PCT/US2022/053097

§ 371 Date: Jun. 14, 2024

Related U.S. Application Data
(60) Provisional application No. 63/290,501, filed on Dec. 16, 2021.

(51) Int. Cl. G16H 50/20; A61B 5/398

(52) U.S. Cl. CPC G16H 50/20; A61B 5/398

(57) ABSTRACT

A method of diagnosing glaucoma using machine learning methods comprises determining a labeled training data set. The labeled training data set comprises electroretinography (ERG) signals measured from a group of subjects. The ERG signals are labeled either glaucomatous or non-glaucomatous based on the subject from which each ERG signal was measured. The training data set is used to train a machine learning model, WNW such as a decision tree model, a discriminant model, a support vector machine, a nearest neighbor algorithm, or an ensemble classifier. The resulting trained machine learning model is configured to classify an ERG signal input as glaucomatous or non-glaucomatous. The model can be employed by measuring an ERG from a subject and inputting the measured ERG into the trained machine learning model. The subject can be diagnosed as having glaucoma based on an output classification of glaucomatous.

### BACKGROUND OF THE INVENTION

Glaucoma, a chronic neurodegenerative disease affecting the retina and optic nerve and a leading cause of blindness, is characterized by progressive, irreversible vision loss. Currently, available treatment paradigms focus primarily on a predisposing factor, elevated intraocular pressure (“IOP”), that does not allow for repair of the retina and optic nerve once the disease has progressed and damage has occurred.

Early-stage glaucoma can be challenging to detect and diagnose. Current technologies for diagnosing glaucoma include various techniques. These techniques include tonometry, involving an instrument that measures the inner pressure of the eye; ophthalmoscopy, involving an instrument that identifies the shape and color of the optic nerve; perimetry, in which a system measures a subject's ability to see an object clearly at several points of their visual field; gonioscopy, in which a lens and lamp are used to distinguish between open-angle and closed-angle glaucoma; and pachymetry, which includes measuring, via a pachymeter, a thickness of the cornea.

### SUMMARY OF THE INVENTION

Introduced here is a framework that uses techniques/technologies to extract relevant features from electroretinography (“ERG”) counts. The framework trains machine learning models using the relevant features to diagnose early-stage glaucoma, distinguish various stages of glaucoma progression, and provide quantitative assessments of visual functionality by predicting retinal ganglion (“RGC”) signals. The framework allows for detecting functional deficits of early/various stages of glaucoma with high accuracy while also providing a more detailed understanding of glaucoma progression and the impact of various therapeutic intervention treatments have on the progression.

Embodiments of the present disclosure are directed to providing mechanisms, including computer-implemented methods and non-transitory computer storage mediums for using ERG signals in a machine-learning-based framework to detect early-stage glaucoma. In some embodiments, the computer-implemented method collects ERG signals that are measurements of the electrical responses of cells in retinas. As the ERG signals are raw data, the method pre-processes the ERG signals by removing anomalies and performing baseline adjustments of the ERG signals. Techniques described herein extract statistical features and advanced wavelet-based features from the pre-processed ERG signals. Using feature extraction techniques, relevant features are selected from the statistical features and the advanced wavelet-based features to create a training dataset. The training dataset can then be used to train machine learning models to make glaucoma-based predictions. These predictions include glaucoma diagnosis prediction, distinguishing between various stages of glaucoma progression, and providing retinal ganglion cell (“RGC”) count predictions that determine a quantitative assessment of the visual functionality of a retina.

At a high level, the technology relates to using machine learning for better detection of early-stage glaucoma. A labeled training data set is determined by measuring ERG signals from a group of subjects and labeling the measured ERG signals as either glaucomatous or non-glaucomatous based on the subject from which each ERG signal was measured. The training data set is used to train a machine learning model, such as a decision tree model, a discriminant model, a support vector machine, a nearest neighbor algorithm, or an ensemble classifier. The resulting trained machine learning model is configured to classify an ERG signal input as glaucomatous or non-glaucomatous.

The trained machine learning model can be employed by measuring an ERG signal from a subject and inputting the measured ERG signal into the trained machine learning model. The subject can be diagnosed with glaucoma based on an output classification of glaucomatous by the trained machine learning model.

This summary is intended to introduce a selection of concepts in a simplified form that is further described in the detailed description section of this disclosure. The summary is not intended to identify key or essential features of the claimed subject matter, nor is it intended as an aid in determining the scope of the claimed subject matter. Additional objects, advantages, and novel features of the technology will be set forth in part in the description that follows and, in part, will become apparent to those skilled in the art upon examination of the disclosure or learned through practice of the technology.

### BRIEF DESCRIPTION OF THE DRAWING

These and other features, aspects, and advantages of the embodiments of the disclosure will become better understood with regard to the following description, appended claims, and accompanying drawings where:

FIG. 1 illustrates a diagram of an architecture for predicting early-stage glaucoma using machine learning in accordance with embodiments of the present disclosure.

FIG. 2 illustrates a diagram of a machine-learning framework predicting early-stage glaucoma using ERG signals in accordance with embodiments of the present disclosure.

FIG. 3 illustrates an example diagnostics system for providing glaucoma diagnoses in accordance with embodiments of the present disclosure.

FIG. 4 illustrates a schematic diagram of a glaucoma diagnosis system in accordance with embodiments of the present disclosure.

FIG. 5 illustrates a flowchart of extracting relevant features from ERG signals to train machine learning models in glaucoma diagnosis in accordance with embodiments of the present disclosure.

FIG. 6 illustrates a flowchart of training a machine learning model and providing a glaucoma diagnosis of an ERG signal in accordance with embodiments of the present disclosure.

FIG. 7 illustrates a flowchart of providing a glaucoma diagnosis through a diagnostics system in accordance with embodiments of the present disclosure.

FIG. 8 illustrates a schematic diagram of an exemplary environment in which a glaucoma diagnosis system and machine learning framework can operate, in accordance with embodiments of the present disclosure.

FIG. 9 illustrates a block diagram of an exemplary computing device, in accordance with an embodiment described herein.

While the present disclosure is amenable to various modifications and alternative forms, specifics thereof, have been shown by way of example in the drawings and will be described in detail. It should be understood, however, that the intention is not to limit the particular embodiments described. On the contrary, the intention is to cover all modifications, equivalents, and alternatives falling within the scope of the present disclosure. Like reference numerals are used to designate like parts in the accompanying drawings.

### DETAILED DESCRIPTION OF THE INVENTION

The present disclosure relates to the detection of early-stage glaucoma and, more specifically, to using electroretinography signals in a machine-learning-based framework to detect early-stage glaucoma. While the present disclosure is not necessarily limited to such applications, various aspects of the disclosure may be appreciated through a discussion of various examples using this context.

### Overview

Glaucoma, a major cause of blindness, is a complex disease that damages the optic nerve resulting in progressive, irreversible loss of eyesight. Early diagnosis of glaucoma can significantly aid the treatment and result in better treatment outcomes and less vision loss. However, existing diagnostic technologies focus either on a surrogate marker for disease development in subjects (intraocular pressure (“IOP”)) or on structural measures. Structural measures are only reliable late in the disease when damage causing vision loss has already occurred and has become irreversible (retinal thickness measured with optical coherence tomography (“OCT”)). Existing technologies do not currently use Electroretinograms (“ERG”) to diagnose glaucoma, as current data analysis methods do not measure parameters relevant to glaucoma and glaucoma disease development. Machine learning methods have great potential in automating glaucoma diagnosis and have been an active area of research focused on analyzing OCT images.

Current technologies for glaucoma diagnosis are based on psychophysical and structural techniques. The former includes Humphrey visual field techniques that use a machine to assess subjects' responses to light stimuli across most of their visual fields, which are still the most commonly utilized techniques for glaucoma diagnosis and monitoring of glaucoma disease progression and efficacy of therapeutic interventions. The latter methods of glaucoma diagnosis include measurements, via an instrument, of retinal structure, specifically the thickness of affected retina layers (e.g., Retinal Nerve Fiber Layer (“RNFL”)) and morphological changes to the part of the retina that connects to the optic nerve (optic nerve head). This field has expanded to include automated retinal image analysis (“ARIA”) systems contributing to diagnosing complex diseases such as diabetic retinopathy and glaucoma. The development of such ARIA systems involved machine learning methods trained with optical coherence tomography (“OCT”) imaging data has led to high analytical accuracy in automatically classifying late-stage glaucoma disease phenotypes based on structural characteristics.

Limitations on glaucoma detection remain with these existing technologies, however, as early detection of glaucoma is still a challenge given the highly significant limitations of early detection of glaucoma based on structural methods. For instance, methods using OCT data are not suitable for the diagnosis of early-stage glaucoma due to their fundamental reliance on structural cell damage, which occurs late in the disease. Additionally, technologies employing analysis of structural changes for glaucoma diagnosis are based on measuring RNFL thickness in OCT images of the retina. This analysis technique can be highly variable and weakly correlated with RGC counts despite RNFL thickness being a surrogate marker of RGC degeneration and optic nerve fiber loss (i.e., hallmarks of glaucoma pathogenesis).

Further, RGC loss often occurs early during pathogenesis without measurable RNFL thinning. Functional measures, such as visual field and ERG testing, provide higher-sensitivity testing methods that are capable of measuring RGC loss earlier during pathogenesis. Functional measures are sensitive to subtle changes in RGC function and RGC damage, which suggest a significant potential to enable early detection of glaucoma, even in the absence of elevated IOP, as seen in subjects with normotensive glaucoma. As a result, glaucoma interventions should be initiated before irreversible damage occurs, allowing for the optimization of treatment strategies based on improving RGC function.

ERG data are complex and multidimensional biomedical data relevant to diagnosing glaucoma but are currently not considered during routine clinical practice or in clinical research. This is due, in part, to multiple barriers related to clinical ERG data acquisition, such as limitations on reproducibility, high costs of both equipment and individual tests, long test duration, complex test administration resulting in reduced subject acceptance and compliance, and the need for highly trained experts to administer the tests.

Newer diagnostic are often time-consuming, labor-intensive, and focused on parameters developed to address a small subset of primarily specific genetic diseases of the eye affecting predominantly pediatric subject populations. Additionally, currently available methods are often unsuitable for analyzing large data sets and databases, rendering them incapable of taking advantage of complex and rich datasets. As a result, early detection of glaucoma is not possible with currently available techniques during the early stages of glaucoma pathogenesis, when cellular changes occur that have yet to result in structural damage or visual impairment.

Embodiments of the present disclosure improve the existing technologies described herein (as well as others) by providing a novel machine-learning framework using ERG signal data for glaucoma detection including early-stage detection. Embodiments of the framework provide technical solutions by extracting and identifying relevant features (i.e., predictors) from ERG signals to train and develop machine learning models to diagnose glaucoma (binary classification), distinguish between various stages of glaucoma progression (multiclass classification) when glaucoma is detected, and provide a quantitative assessment of visual function by predicting RGC counts from the ERG signals.

Embodiments disclosed herein take advantage of changes in the activity of the retinal nerve cells most affected by glaucoma, RGC, an early feature of glaucoma development and an aspect of early pathogenesis that often occurs in the absence of measurable RNFL thinning. While retina function can be measured with ERGs, the analysis of subtle changes in RGC activity indicative of glaucoma cannot be measured with currently available commercial methods for ERG analysis prompting an urgent clinical need for higher sensitivity methods. ERG signals that are sensitive to subtle changes in RGC function due to glaucoma as reliably detected by the disclosed technology and enable early detection of glaucoma, even in the absence of elevated IOP or thinning of the retina and RNFL.

More specifically, embodiments improve upon existing technologies by developing predictive models for early-stage glaucoma diagnosis based on machine-learning algorithms utilizing relevant features from ERG signals as predictors. The glaucoma framework provides novel techniques that pre-process the ERG signal data, extract relevant features of the ERG signal data, and train machine learning models using the relevant features. The resulting machine learning models have the technical effect of achieving higher accuracy of early-stage glaucoma diagnosis while also providing a more detailed understanding of disease progression and provide a better understanding of the impact of therapeutic interventions relative to existing technologies.

In some embodiments, the machine learning framework trains a glaucoma diagnosis machine learning (“ML”) model using the relevant features extracted from ERG signals. The glaucoma diagnosis ML model is trained to provide a binary classification (i.e., classifying glaucomatous or non-glaucomatous) based on ERG signals used as input. In some implementations, the machine learning framework uses relevant statistical features in making its prediction. The relevant statistical features include a correlation of cones, mean and median of flicker, skewness of Hi-Rods and cones, and standard deviation of cones. Through various implementations, the glaucoma diagnosis ML model can be configured as a decision-tree model, a discriminant classifier, a support vector machine (“SVM”) model, an ensemble classifier model, or a Naive Bayes model.

In some embodiments, the machine learning framework trains a glaucoma progression ML model using the relevant features extracted from ERG signals. The glaucoma progression ML model is trained to provide a multiclass classification (i.e., classifying the various progression/stages of glaucoma) based on ERG signals used as input. In some implementations, the glaucoma progression ML model uses statistical features in its multiclass prediction. The statistical features include correlation of cones, number of troughs in Hi cones, kurtosis of scotopic threshold response (“STR”), and mean of flicker. In some implementations, the glaucoma progression ML model uses advanced wavelet-based features in making its prediction. These advanced wavelet-based features include wavelet variance of rods and Shannon Entropy Values and AR coefficients for Maximal Overlap Discrete Wavelet Packet Transform (“MOD-PWT”). Through various implementations, the glaucoma progression ML model is configured as a decision-tree model, a discriminant classifier, a support vector machine (“SVM”) model, an ensemble classifier model, or a Naive Bayes model.

In some embodiments, the glaucoma diagnosis framework trains an RGC count ML model using the relevant features extracted from ERG signals. The RGC count model is trained to provide a classification (i.e., classifying the RGC count) based on ERG signals used as input. In some implementations, the RGC count model uses a regression analysis (e.g., a Gaussian Process Regression) to predict the RGC count from the ERG signals. In some other implementations, the machine learning model is an artificial neural network (“ANN”). In some embodiments, the machine learning framework uses mRmR sequential feature selection when determining the feature selection for the regression analysis.

In some embodiments, the glaucoma diagnosis framework includes pre-processing raw ERG signals. ERG raw data can contain several anomalies, such as different start times, missing data, different sampling frequencies, noise, unequal lengths of signal recordings, and the like. In order to account for the anomalies, the glaucoma diagnosis framework provides pre-processing measures, including performing baseline adjustments, feature extraction, missing data correction, outlier correction, feature scaling, feature selection, and the like.

In some embodiments, the glaucoma diagnosis framework extracts statistical features from the ERG signals. The statistical features include, but are not limited to, measures of central tendency, spread, shape, peaks, derivatives, and correlations extracted from the ERG signals. These statistical features are capable of describing the general behavior of ERG signals.

In some embodiments, the glaucoma diagnosis framework extracts advanced wavelet-based features using an autoregressive model. The autoregressive model can describe specific time-varying processes and specifies that an output variable depends linearly on its previous values and a stochastic term. In some implementations, the autoregressive model operates under the premise that past value have an effect on current values.

In some embodiments, the glaucoma diagnosis framework extracts advanced wavelet-based features using Shannon entropy. Shannon entropy is an information-theoretic measure of a signal Shannon entropy values for the MOD-PWT using four-level wavelet decomposition computed on terminal nodes of the wavelet.

In some embodiments, the glaucoma diagnosis framework extracts advanced wavelet-based features using multifractal wavelet leader estimates and cumulant scaling exponents. Wavelet leaders are time/space-localized suprema of the discrete wavelet coefficients' absolute value. These suprema are used to calculate the Holder exponents, which characterize the local regularity. Additionally, the second cumulant of the scaling exponents is obtained. Scaling exponents are scale-dependent exponents that describe a signal's power-law behavior at various resolutions.

The techniques described herein provide various technical improvements over conventional methods of glaucoma diagnosis. For example, embodiments that provide mechanisms for binary classification of glaucoma diagnosis provide higher accuracy of diagnosis by utilizing relevant features extracted from ERG signals. These mechanisms allow users to provide ERG signals of a subject to retrieve a glaucoma diagnosis with higher accuracy over conventional methods. Additionally, these mechanisms also provide accurate diagnoses of early-stage glaucoma to allow for treatments to occur before permanent damage to a retina occurs. Furthermore, embodiments describing techniques to extract relevant features from ERG signals also allow for the training of machine learning models to provide predictions capable of distinguishing various stages of glaucoma progression. Machine learning models also provide quantitative assessments of visual function by predicting RGC counts.

### Example Machine Learning Framework

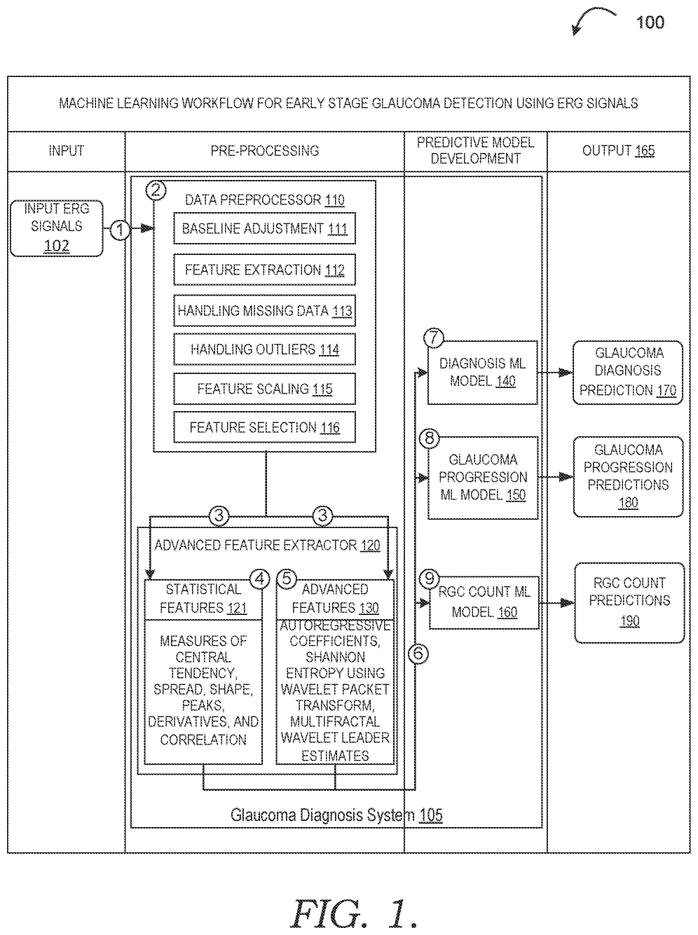

FIG. 1 illustrates a diagram of a machine learning framework 100 implementing a glaucoma diagnosis system 105 configured to extract relevant features from ERG signals and train machine learning models in glaucoma detection and diagnosis, in accordance with embodiments of the present disclosure. As discussed, the glaucoma diagnosis system 105 uses a plurality of machine learning models to diagnose glaucoma, including early stages of glaucoma, distinguish various stages of glaucoma progression, and provide a quantitative assessment of visual functionality of retinas by predicting RGC count from ERG signals. The machine learning predictions allow the glaucoma diagnosis system 105 to achieve higher accuracy and provide a more detailed understanding of disease progression over current conventional methods of glaucoma diagnosis using the novel techniques described herein. As such, the glaucoma diagnosis system 105 provides glaucoma diagnosis predictions using an ERG signal of a subject once the machine learning models are trained with a training dataset with relevant features extracted from ERG signals. During application, if the presence of glaucoma is predicted, additional machine learning models can also predict the current glaucoma progression and the RGC count of the subject.

The machine learning framework 100 and/or the glaucoma diagnosis system 105 can be implemented as a standalone application or as part of another application or suite of applications. For example, in some embodiments, the machine learning framework 100 and/or the glaucoma diagnosis system 105 is implemented as part of a diagnostics application, enabling the diagnosis and status of glaucoma of a subject by a user of a diagnostics application.

Alternatively, once a subject receives a glaucoma diagnosis, the glaucoma diagnosis system 105 can utilize the ERG signals of the subject to provide a stage of glaucoma progression and/or RGC count of the subject.

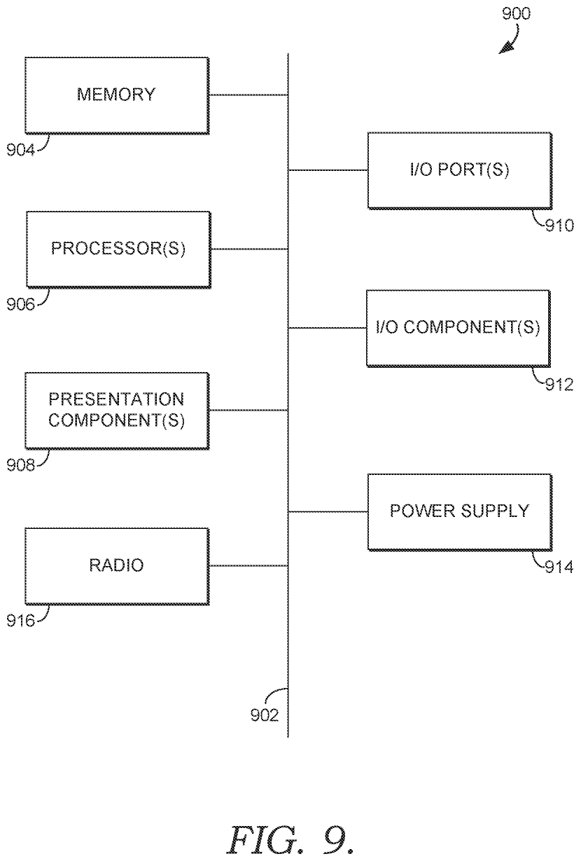

The glaucoma diagnosis framework 100 includes input ERG signals 102, a glaucoma diagnosis system 105, and output 165. The glaucoma diagnosis system 105 includes data preprocessor 110, an advanced feature extractor 120, a glaucoma diagnosis ML model 140, a glaucoma progression ML model 150, and an RGC count ML model 160. The output 165 includes glaucoma diagnosis predictions 170, glaucoma progression predictions 180, and RGC count predictions 190. As shown, FIG. 1 provides an example method that may be performed by the computing device of FIG. 9 and is suitable for achieving the described advantages and detecting early-stage glaucoma. In embodiments, one or more computer storage media having computer-executable instructions embodied thereon that, when executed, by one or more processors, cause the one or more processors to perform operations illustrated in FIG. 1.

As shown in FIG. 1, the glaucoma diagnosis system 105 receives input ERG signals 102 at numeral 1. As discussed further below, the glaucoma diagnosis system 105 trains a plurality of machine learning models to provide glaucoma diagnosis predictions, glaucoma progression predictions, and RGC count predictions using a plurality of ERG signals converted into training data. The input signals 192 may comprise ERG signals measured from subjects and associated with labels. Each ERG signal can be associated with a label of glaucomatous or non-glaucomatous. The subjects may include animal subjects, such as mammalian subjects, including mice and the like, as well as human subjects.

The ERG signals include measurements such as Oscillatory Potential (“OP”) and Scotopic Threshold Response (“STR”) that represent important ERG components indicative of RGC cell function. OPs are small rhythmic wavelets superimposed on the ascending b-wave of an ERG signal. STRs are negative corneal deflections elicited in the fully dark-adapted eye to dim stimuli. An International Society for Clinical Electrophysiology of Vision (“ISCEV”) standardized ERG protocol that includes several tests to measure the function of various retinal cell types. These tests include rod response, standard rod-cone response, Hi-intensity rods, cone response, Hi-intensity cone response, flicker, and Hi flicker.

At numeral 2, the data preprocessor 110 implements data preprocessing techniques such as baseline adjustment 111, feature extraction 112, handling missing data 113, handling outliers 114, feature scaling 115, and feature selection 116 on the input ERG signals 102. At numeral 3, the preprocessed ERG signals are provided to the advanced feature extractor 120 to extract statistical features 124 and advanced wavelet-based features 126. Unlike prior techniques that utilized morphological and transitional characteristics of ERG signals, embodiments utilize statistical and wavelet-based features to train machine learning models in glaucoma diagnosis prediction. Particularly, embodiments train machine learning models to predict early-stage glaucoma when cellular changes occur that have yet to result in structural damage or visual impairment. As discussed, this includes training a glaucoma diagnosis ML model 140 to provide a binary classification predicting whether glaucoma is present using the relevant features (e.g., statistical and wavelet-based features) extracted from the ERG signals.

For example, the advanced feature extractor 120 includes statistical feature techniques 124 and advanced wavelet-based feature techniques 126 that extract the features from the preprocessed ERG signals. As discussed further below, this can include statistical features such as measures of central tendency, spread, shape, peaks, derivatives, and correlation at numeral 4. This also includes advanced wavelet-based features such as autoregressive coefficients, Shannon entropy, and multifractal wavelet leader estimates at numeral 5.

In various embodiments, the resulting relevant features output by the advanced feature extractor 120, in the form of training datasets, are then provided to the ML models 140, 150, 160, respectively, at numeral 6. The glaucoma diagnosis ML 140 model can be trained using ML training techniques, such as supervised learning, to provide a binary classification predicting a glaucoma diagnosis 170 at numeral 7. The glaucoma progression ML model 150 can be trained using ML techniques to provide a multiclass classification predicting a glaucoma progression 180 at numeral 8. The RGC count ML model 160 can be trained using ML techniques to provide a classification predicting an RGC count 190.

In various embodiments, the ML models described herein can be implemented as several types of machine learning models. These models include, but are not limited to, decision trees, discriminant, support vector machine, nearest neighbor, and ensemble classifiers. These machine learning models can perform both classification and regression. Decision tree-based models predict the target variable by learning decision rules. Discriminant classifiers are based on the assumption that each class has different Gaussian distribution of data, and the classifications are performed based on Gaussian distribution parameters estimated by the fitting function. Support vector machine (“SVM”) is based on Vapnik-Chervonenkis theory, where a hyperplane separating the classes is determined. SVMs are efficient algorithms suitable for compact datasets. The nearest neighbor algorithm is based on the assumption that similar things exist nearby. Ensemble methods such as bagged trees (or random forest), combine the predictions of several learning algorithms with improving generalization. Regression analysis, based on the above techniques, can also be performed alongside classification. Regression analysis is a set of statistical processes for estimating the relationships between a dependent variable (e.g., the label in a training dataset) and one or more independent variables (also referred to as predictors or features).

In various embodiments, the ML models described herein may be implemented as multilayer perceptrons (“MLPs”), convolutional neural networks (“CNNs”), or other neural networks. Alternatively, some neural networks may be implemented as MLPs while others are implemented as CNNs or other combination of neural networks. A neural network can include a machine learning model that can be tuned (e.g., trained) based on training input (e.g., relevant features extracted from ERG signals) to approximate unknown functions. In particular, a neural network can include a model of interconnected digital neurons that communicate and learn to approximate complex functions and generate outputs based on a plurality of inputs provided to the model. For instance, the neural network includes one or more machine learning algorithms. In other words, a neural network is an algorithm that implements deep learning techniques, i.e., machine learning that utilizes a set of algorithms to attempt to model high-level abstractions in data.

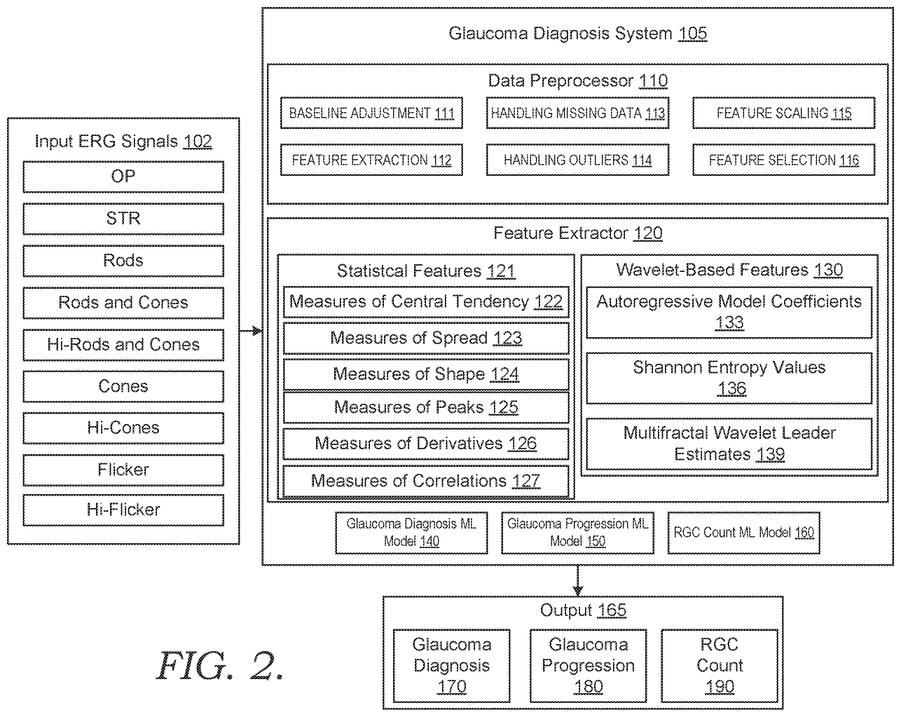

FIG. 2 illustrates a diagram of a machine learning framework 100 for glaucoma diagnosis using machine learning models, in accordance with embodiments of the present disclosure. As discussed, embodiments use preprocessing techniques and feature extraction methods to produce unique and relevant features extracted from ERG signals to train ML models in glaucoma diagnosis. As shown, the machine learning framework 100 includes input ERG signals 102, a glaucoma diagnosis system 105, and output 165. The glaucoma diagnosis system includes a data preprocessor 110, an advanced feature extractor 120, a glaucoma diagnosis ML model 140, a glaucoma progression model 150, and an RGC count ML model 160. This combination of components enables the machine learning framework 100, through the glaucoma diagnosis system 105, to train machine learning models to provide glaucoma-related diagnoses given ERG signals of a subject.

While previous techniques utilized neural networks to automate glaucoma diagnosis based on ERG signals, they merely utilized morphological and transitional characteristics of ERG signals as features (thirteen features) for training their model. These methods were limited to basic morphological characteristics of mfERG recordings. Previous techniques also employed neural networks for ERG-based glaucoma diagnosis but used continuous wavelet-transformed coefficients. This prior technique was limited to wavelet features only. In contrast, embodiments utilize several advanced features extracted from ERG signals beyond morphological and transitional characteristics, as described further down in more detail, to provide higher accuracy and earlier detection of glaucoma in subjects.

The input ERG signals 102 include measurements of electrical responses of different types of cells in retinas. These measurements include, but are not limited to, rod responses (dark-adapted 0.01 ERG), maximal or standard combined rod-cone response (dark-adapted 3.0 ERG), OP (dark-adapted 3.0 oscillatory potentials, single-flash cone response (light-adapted 3.0 ERG), flicker (light-adapted 3.0 flicker ERG), macular or focal ERG, multifocal ERG, pattern ERG, early receptor potential, STR, direct-current ERG, long-duration light-adapted ERG (on-off responses), double-flash ERG, chromatic stimulus ERG (s-cone ERG), a dark and light adaptation of the ERG, dark-adapted and light-adapted luminance-response analyses, and saturated a-wave slope analysis.

Stimulus (and response) names can be described by the state of light adaptation, and the flash strength represented as cd·s·m⁻². For example, the dark-adapted response to 3.0 cd·s·m⁻² is called the “Dark-adapted 3.0 ERG”. In addition, descriptive terms (such as “rod response”, “mixed rod-cone response”, etc.) can be used. This scheme of naming is also applied to non-standard stimuli, which might be used for special protocols or because of equipment limitations (e.g., if flashes of 15.0 cd·s·m⁻² are used under dark-adapted conditions, they can be specified as a “dark-adapted 15.0 ERG”).

Each ERG signal can include representative waveforms of each standard ERGs displayed with amplitude and time calibrations. The ERG signals can also be labeled with stimulus variables and the state of light and dark adaptation. In some implementations, the ERG signals include at least 20 milliseconds of baseline prior to the stimulus for single flash responses and indicate the stimulus for each flash with a mark or line. Two responses from each stimulus condition can be displayed to demonstrate the degree of consistency or variability.

The glaucoma diagnosis system 105 is a component of the machine learning framework 100 configured to develop training datasets with relevant features extracted from the input ERG signals 102 and to train machine learning models using the relevant features. The glaucoma diagnosis system 105 develops the glaucoma diagnosis ML model 140 for early glaucoma diagnosis based on machine learning algorithms by utilizing the advanced relevant features from the input ERG signals 102 as predictors. First, the data preprocessor 110 preprocesses the input ERG signals 102, and the feature extractor 120 extracts the advanced relevant features (e.g., statistical features 121, advanced wavelet-based features 130).

The data preprocessor 110 is a component of the glaucoma diagnosis system 105 configured to preprocess the input ERG signals 102. Preprocessing transforms raw data into a usable format by removing anomalies such as different start times, missing data, different sampling frequencies, noise, and unequal lengths of signal recordings. In some implementations, the data preprocessor 110 performs baseline adjustments 111 on the input ERG signals as part of the preprocessing process. An ERG signal's baseline (start time) can be different for different subjects and testing protocols. Therefore, during baseline adjustment 111, the measurements can be brought to a common baseline (start time offset to zero) during baseline adjustment. In some embodiments, the data preprocessor 110 performs baseline adjustment techniques such as median filter, linear phase high pass filter, and mean median filter to perform baseline adjustments 111 on the input ERG signals 102.

In some implementations, the data preprocessor 110 performs feature extraction 112, and feature selection, on the input ERG signals as part of the preprocessing process. Feature extraction involves computing a reduced set of values from a high-dimensional signal capable of summarizing most of the information contained in the signal. Feature extraction techniques develop a transformation of the input space onto the low-dimensional subspace that attempts to preserve the most relevant information. In feature selection, input dimensions that contain the most relevant information for solving a particular problem are selected. These methods aim to improve performance, such as estimated accuracy, visualization, and comprehensibility. An advantage of feature selection is that important information related to a single feature is not lost, but if a small set of features is required and original features are very diverse, there is chance of information being lost as some of the features must be omitted. On the other hand, with dimensionality reduction, also known as feature extraction, the size of the feature space can often be decreased without losing information about the original feature space.

In some implementations, the data preprocessor 110 applies feature extraction techniques to perform some transformation of original features to generate other features that are more significant. These feature extraction techniques include, but are not limited to Minimum Redundancy Maximum Relevance (“mRmR”), Relief, Conditional Mutual Information Maximization (“CMIM”), Correlation Coefficient, Between-Within Ratio (“BW-ratio”), Interact, Genetic Algorithms (“GA”), Support Vector Machine-Recursive Feature Elimination (“SVM-REF”), Principal Component Analysis (“PCA”), Non-Linear Principal Component Analysis, Independent Component Analysis, and Correlation based feature selection. These feature extraction techniques are useful for machine learning because they can reduce the complexity of input data and give a simple representation of data representing each variable in feature space as a linear combination of the original input variable.

In some embodiments, the data preprocessor performs missing data techniques 113 for handling gaps in the input ERG signals 102. These missing data techniques 113 include complete case analysis, single imputation, log-linear models and estimation using the EM algorithm, propensity score matching, and multiple imputations. The technique confines attention to cases for which all variables are observed in a complete case analysis. In a single implicit imputation method, missing values are replaced by values from similar responding units in the sample. The similarity is determined by looking at variables observed for both respondent and non-respondent data. Multiple imputations replace each missing value with a vector of at least two imputed values from at least two draws. These draws typically come from stochastic imputation procedures. In the log linear model, cell counts of a contingency table are modeled directly. An assumption can be that, given expected values for each cell, the cell counts follow independent multivariate Poisson distributions. These are conditional on the total sample size, with the counts following a multinomial distribution.

In some embodiments, the data preprocessor 110 performs outlier detection and correction techniques for handling outlier data within the input ERG signals 102. Outliers, by virtue of being different from other cases, usually exert a disproportionate influence on substantive conclusions regarding relationships among variables. An outlier can be defined as a data point that deviates markedly from other data points.

For example, error outliers are data points that lie at a distance from other data points because they result from inaccuracies. More specifically, error outliers include outlying observations that are caused by not being part of the targeted population of data, lying outside the possible range of values, errors in observation, errors in recording, errors in preparing data, errors in computation, errors in coding, or errors in data manipulation. These error outliers can be handled by adjusting the data points to correct their values or more such data points from the data set. In some implementations, the data preprocessor 110 defines values more than three scaled median absolute deviations (“MAD”) away from the median as outliers. Once defined as an outlier, the data preprocessor 110 replaces the values with threshold values used in outlier detection.

In some embodiments, the data preprocessor 110 performs feature scaling 115 on the input ERG signals 102 as part of the data preprocessing process. Feature scaling is a method to unify self-variables or feature ranges in data. Feature scaling is a necessary step in the calculation of stochastic gradient descent. The data preprocessor 110 can perform various feature scaling 115 techniques. These feature scaling 115 techniques include, but are not limited to, data normalization methods and interval scaling.

Data normalization is a basic work of data mining. Different evaluation indicators often have different dimensions, and the difference in numerical values may be very large. Without processing, the results of data analysis may be affected. Standardized processing is needed in order to eliminate the influence of dimension and range differences between indicators. The data is scaled to a specific area to facilitate comprehensive analysis. The premise of the normalization method is that the eigenvalues obey the normal distribution, and each genus is transformed into a standard positive distribution with a mean of 0 and a variance of 1 by translation and scaling data transformation. The interval method utilizes the boundary information to scale the range of features to a range of features. For example, the commonly used interval scaling methods such as [0, 1] use two extreme values (maximum and minimum values) for scaling.

In some embodiments, the data preprocessor 110 perform feature selection 116 techniques on the input ERG signals 102 for dimensionality reduction from the extracted features. The feature selection 116 techniques can be used to reduce the computational cost of modeling, to achieve a better generalized, high-performance model that is simple and easy to understand. Feature extraction technique 112, as discussed previously, can be performed to reduce the signals' dimensionality. However, in some implementations, the resulting number of features may still be higher than the number of training data. Therefore, further reduction in the dimensionality of the data can be performed using feature selection 116 techniques to identify relevant features for classification and regression. Feature selection 116 techniques can reduce the computational cost of modeling, prevent the generation of a complex and over-fitted model with high generalization error, and generate a high-performance model that is simple and easy to understand. In some embodiments, the data preprocessor 110 uses the mRmR sequential feature selection algorithm to perform feature selection 116. The mRmR method is designed to drop redundant features, which can design a compact and efficient machine learning-based model.

The advanced feature extractor 120 is a component of the glaucoma diagnosis system 105 configured extract advanced relevant features from the input ERG signals. The advanced relevant features can capture subtle changes in a retina that indicate potential signs of glaucoma. The advanced feature extractor 120 is configured to extract features in two phases. First, statistical features 121 can be extracted from the input ERG signals 102, followed by extracting advanced wavelet-based features 130.

In some embodiments, the statistical feature extraction 121 techniques produce statistical features capable of describing the general behavior of ERG signals extracted from the input ERG signals 102. These features include, but are not limited to, measures of central tendency, spread, shape, peaks, derivatives, and correlation. Measures of central tendency included mean, median, trimmed mean. Measures of spread included range, standard deviation, variance, mean absolute deviation, and interquartile range. Measures of the shape include skewness, kurtosis, central moments of the second and third order, and aspect ratio. Measures of peaks included the number of peaks and troughs in the signal. Measures of derivatives include the first-order derivative of the signal with respect to time. Measures of correlation included the correlation coefficient of the signal with respect to time.

In some embodiments, the advanced feature extractor 120 uses an autoregressive (“AR”) coefficients process to extract the advanced wavelet-based features. Provided a signal x[n] at time instant n in an AR process of order p can be described as a linear combination of p earlier values of the same signal. In some implementations, the AR coefficients procedure is modeled as follows:

$$x [ n ] = {\sum }_{i = 1}^{p} a [ i ] x [ n - i ] + e [ n ]$$

where a[i] is the AR model's ich coefficient, e[n] denotes white noise with mean zero, and p denotes the AR order. In some implementations, the AR coefficients for each block are estimated using the Burg method. The order can be determined using the ARfit model order selection method as fourth order. As such, a 4-order AR model can be chosen to represent each of the ERG signal components.

In some embodiments, the feature extractor 120 uses wavelet-based Shannon entropy to extract the advanced wavelet-based features. Shannon entropy is an information-theoretic measure of a signal. Shannon entropy values for the MOD-PWT using four-level wavelet decomposition can be computed on terminal nodes of the wavelet.

Shannon entropy, or information entropy, measures how much information there is in an event. In general, the more certain or deterministic the event is, the less information it will contain. More clearly stated, information is an increase in uncertainty or entropy. As an example, suppose someone is told something they already know, making the information they receive very small. As such, this information would have very low entropy. If they were told about something they knew little about, they would receive a lot of new information. As such, this information would have high entropy. In the context of wavelet packets, in some implementations, the mathematical expression for Shannon entropy using wavelet packet transform is as follows:

$$S {E}_{j} = - {\sum }_{k = 1}^{N} {p}_{j , k} * log {p}_{j , k}$$

where N is the number of coefficients in the j^{th} node, and p_{j,k} are the normalized squares of the wavelet packet coefficients in the j^{th} terminal node of the wavelet.

In some embodiments, the advanced feature extractor 120 uses multifractal leader estimates 139 and multiscale wavelet variance estimate to extract the advanced wavelet-based features. In some implementations, the multifractal measure of the ERG signals is obtained using two wavelet methods (wavelet leader and cumulant of the scaling exponents). Wavelet leaders are time/space-localized suprema of the discrete wavelet coefficients' absolute value. These suprema are used to calculate the Holder exponents, which characterize the local regularity. In addition, a second cumulant of the scaling exponents can be obtained. Scaling exponents are scale-dependent exponents that describe the signal's power-law behavior at various resolutions. The second cumulant can depict the scaling exponents' divergence from linearity. Wavelet variance of ERG signals can also be obtained as features. Wavelet variance quantifies the degree of variability in a signal by scale, or more precisely, the degree of variability in a signal between octave-band frequency intervals.

The glaucoma diagnosis ML model 140 is a component of the glaucoma diagnosis system 105 configured to predict binary classifications of glaucoma diagnosis (e.g., glaucomatous and non-glaucomatous). The glaucoma diagnosis ML model 140 is trained using a training dataset compiled by the data preprocessor, including the relevant features extracted by the feature extractor 120. The glaucoma diagnosis ML model 140 can be configured as several different types of machine learning models that can be trained to classify the ERG signals. These machine learning models include, but are not limited to, decision trees, discriminant, SVM, nearest neighbor, ensemble classifiers, and neural networks. In some embodiments, the binary classification produced by the glaucoma diagnosis ML model 140 is based on statistical features including the correlation of cones, mean of flicker, median, and skewness of Hi rods and cones, and standard deviation of cones. In some embodiments, the binary classification produced by the glaucoma diagnosis ML model 140 is based on wavelet-based features. These extracted wavelet features include Shannon entropy values for MOD-PWT, Rods and cones, Rods, STR, and OP.

The glaucoma progression ML model 150 is a component of the glaucoma diagnosis system 105 configured to provide a multiclass classification of glaucoma progression. In some embodiments, the glaucoma progression classification is based on IOP as (normal, <12 mm Hg; high, [≥12 mm Hg<17 mm Hg]; glaucomatous, ≥17 mm Hg). In some implementations, multiclass classification (classifying different stages, normal, high, and glaucomatous) is based on statistical features extracted by the feature extractor 120. These statistical features include the correlation of cones, number of troughs in Hi cones, kurtosis of STR and mean of flicker. The glaucoma progression ML model 150 can be configured as several different machine learning models. These machine learning models include, but are not limited to, SVM and ensemble-based classifiers (e.g., bagged trees model).

In some implementations, the multiclass classification is performed using wavelet-based features. These extracted wavelet features include wavelet variance of rods and Shannon Entropy Values. AR coefficients for MOD-PWT can be used as features from Hi-Flicker, Flicker, Hi-cones, and STR. The identification of the flicker ERG test and the corresponding features, among other tests, reconfirmed the capability of the current approach in identifying the relevant features and can assist in early-stage diagnosis of glaucoma. Using these approaches provides an improvement in accuracy over conventional methods that indicate that wavelet-based features can distinguish healthy and glaucomatous subjects suggesting that they are more sensitive to subtle changes in ERG signals due to glaucoma. The multiclass classification ability of the glaucoma diagnosis system 105 reaffirms the complex nature of ERG signals in assessing the disease progression.

The RGC count ML model 160 is a component of the glaucoma diagnosis system 105 configured to predict and RGC count based on ERG signals. In some embodiments, the RGC count ML model 160 uses regression machine learning techniques to predict an RGC count from ERG signals. These regression machine learning techniques include, but are not limited to, Gaussian Process Regression (“GPR”), linear regression, decision tree, support vector regression, lasso regression, and random forest. In some embodiments, feature selection for the RGC count ML model 160 is performed using mRmR sequential feature selection and trained with statistical features and wavelet-based features, individually and/or collectively. In some implementations, the resulting trained RGC count ML model 160 indicates that RGC counts strongly correlate with STR and OPs, making them the dominant features selected for RGC regression.

In some embodiments, the glaucoma diagnosis system 105 evaluates the performance of the ML models 140, 150, and 160 during the training process. Various performance evaluation metrics can be utilized to compare the performance of different machine learning algorithms. These metrics include, but are not limited to, accuracy, sensitivity, specificity, precision, recall, f-score, and root mean squared error.

In some implementations, the following mathematical formulations can be used to calculate the different evaluation metrics. True Positive (“TP”) can represent cases when the models correctly predicted a positive (i.e., glaucomatous) class. True Negative (“TN”) can represent cases when the models correctly predicted the negative (i.e., non-glaucomatous) class. False Positive (“FP”) can represent the cases when the models incorrectly predicted the positive (i.e., glaucomatous) class. False Negative (“FN”) can represent the cases when the models incorrectly predicted the negative (i.e., non-glaucomatous) class. Accuracy is the percentage of correctly classified observations, as calculated below:

$$Accuracy ( \% ) = \frac{T P + F P}{T P + T N + F P + F N}$$

Sensitivity/Recall can estimate the proportion of actual positives (e.g., actual glaucomatous) identified as calculated below:

$$Sensitvity / Recall ( R E ) = \frac{T P}{T P + F N}$$

where recall estimates a model's ability to correctly reject healthy subjects without a glaucoma.

Precision can estimate the proportion of positive predictions (e.g., glaucomatous predictions) that are correct, as calculated below:

$$Precision ( PR ) = \frac{T P}{TP + FP}$$

The F-score can estimate the harmonic mean of the precision and recall, as calculated below:

$$F - Score = \frac{PR \times RE}{P R + R E}$$

The Root Mean Square Error (“RMSE”) can also be used as a performance evaluation metric for regression analysis (e.g., analysis for the RGC count ML model 160). In some implementations, RSME is the standard deviation of the prediction errors (residuals), as calculated below:

$$RMSE = \sqrt{\frac{{\sum }_{i = 1}^{N} {( {Actual}_{{x}_{i}} - {Predicted}_{{\hat{x}}_{i}} )}^{2}}{N}}$$

where N is the number of observations.

It is noted that FIG. 2 is intended to depict the major representative components of a machine learning framework 100. In some embodiments, however, individual components may have greater or lesser complexity than as represented in FIG. 2, components other than or in addition to those shown in FIG. 2 may be present, and the number, type, and configuration of such components may vary.

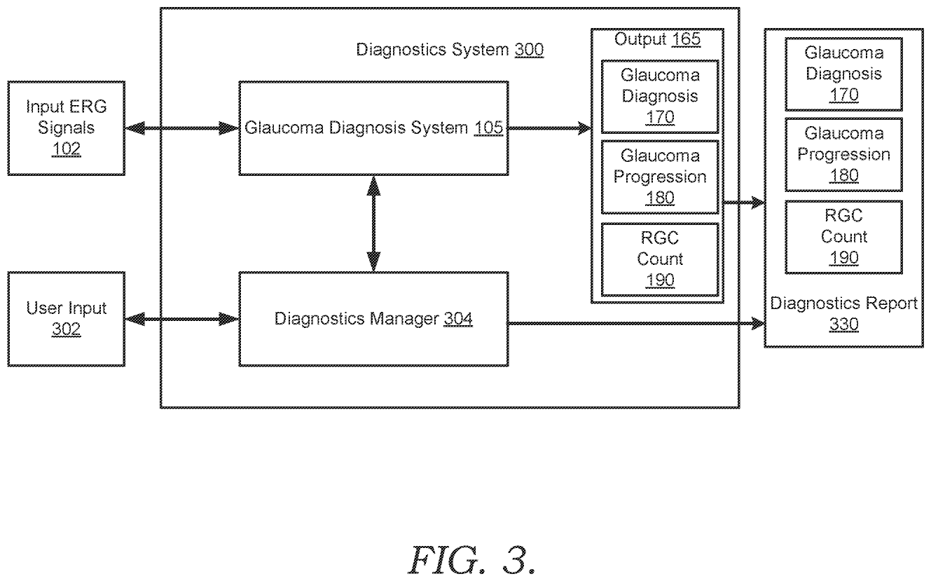

FIG. 3 illustrates an example diagnostic evaluation of an ERG signal using a diagnostics system 300, in accordance with the embodiments of the present disclosure. As discussed, techniques described herein provide a glaucoma diagnosis 170 (e.g., glaucomatous, non-glaucomatous), a glaucoma progression diagnosis 180 (e.g., normal, high, and glaucomatous), and an RGC count analysis 190. Accordingly, in some embodiments, a user can provide ERG signal data 102 to train the ML models 140, 150, 160 of the glaucoma diagnosis system 105 to generate a glaucoma diagnostics report 330 that can include the glaucoma diagnosis 170, glaucoma progression 180, and RGC count 190. As shown in FIG. 3, optionally, the glaucoma diagnosis system 105 is implemented as part of the diagnostics system 300. Alternatively, the glaucoma diagnosis system 105 is implemented in a separate system, which provides at least the output 165 to the diagnostics system 300 to be evaluated by the user.

Once the output 165, representing the glaucoma predictions of an ERG signal, have been obtained by the diagnostics system 300, the output 165 can be evaluated by the user (e.g., medical professional). In some implementations, user input 302 (e.g., ERG signal data) can be received by a diagnostics manager 304 of the diagnostics system 300. The user input 302 can include any information regarding a potential subject, including their ERG signal data. The diagnostics manager 304 can generate a diagnostics report 330 including diagnostics known in the art and may include the output 165 produced by the glaucoma diagnosis system 105 as a result of evaluating the user input 302.

In some implementations, the resulting classification output 165 generated by the glaucoma diagnosis system 105 may be output to a different or downstream diagnostics system for evaluation and responsive action. The responsive action may take any known or later developed form, including output, to a medical professional, such as via a user interface, when the ERG signal data indicates a glaucomatous diagnosis. The user interface may comprise user interface elements for drilling down into the details of the notification, including identifying the specific ERG signal data of a subject and their corresponding classifications produced by the corresponding ML model 140, 150, and 160. In this way, the medical professional may identify which features of the ERG signal data contributed to the diagnosis as well as the progression of the glaucomatous state. Moreover, probability/confidence values, weighting values, and the like, for the classifications may be provided in the notification to indicate more basis for the classifications associated with the ERG signal data. User interface elements may be provided for allowing the medical professional to provide input to indicate a correctness/incorrectness of the classifications of the ERG signal data such that this information may be stored for creation of new training datasets for updating the training of the ML models 140, 150, 160 at a future time.

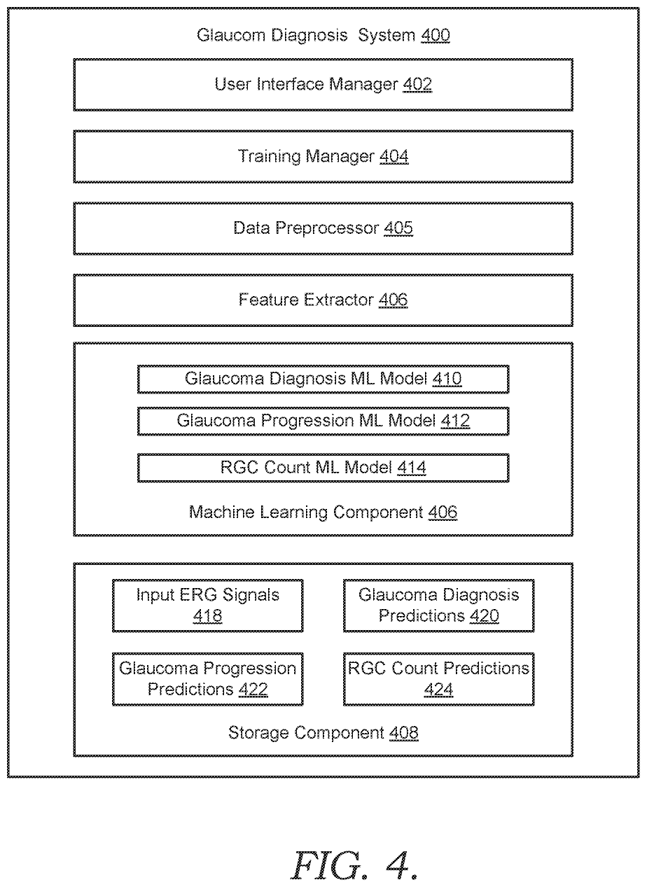

FIG. 4 illustrates a schematic diagram of a glaucoma diagnosis system 400 (e.g., “the glaucoma diagnosis system 105” described above), in accordance with embodiments of the present disclosure. As shown, the glaucoma diagnosis system 400 includes, but is not limited to, a user interface manager 402, a training manager 404, a data preprocessor 405, a machine learning component 406, and a storage manager 408. The machine learning component 406 includes a glaucoma diagnosis ML model 410, a glaucoma progression ML model 412, and an RGC count ML model 414. The storage manager 408 includes input ERG signals 418, glaucoma diagnosis predictions 420, glaucoma progression prediction 422, and RGC count predictions 424.

As illustrated in FIG. 4, the glaucoma diagnosis system 400 includes a user interface manager 402. For example, the user interface manager 402 allows users to input ERG signals 418 to the glaucoma diagnosis system 400. In some embodiments, the user interface manager 402 provides a user interface through which the user can upload the input ERG signals 418 representing the ERG signal data of a subject, as discussed above. Alternatively, or additionally, the user interface may enable the user to download the input ERG signals 418 from a local or remote storage location (e.g., by providing an address (e.g., a URL or other endpoint) associated with an input ERG signal 418 sources). In some embodiments, the user interface can enable a user to link an ERG signal capture device, such as electroretinography equipment or other hardware, to capture ERG signal data and provide it to the glaucoma diagnosis system 400. In some embodiments, the user interface manager 402 also enables the user to provide specific ERG signal data to be evaluated and analyzed. Additionally, the user interface manager 402 allows users to request the glaucoma diagnosis system 400 to provide specific diagnostic predictions. For example, a user can request just a glaucoma diagnosis prediction. In some embodiments, the user interface manager 402 enables the user to edit the input ERG signal data 418. Alternatively, the input ERG signals 418 can be evaluated in a separate diagnostics system separate from glaucoma diagnosis system 400, as discussed above.

As illustrated in FIG. 4, the glaucoma diagnosis system 400 also includes a training manager 704. The training manager 704 can teach, guide, tune, and/or train one or more machine learning models. In particular, the training manager 704 can train a machine learning model based on a plurality of training data (e.g., input ERG signals 418). As discussed, the input ERG signals 418 includes ERG signal data captured from various subjects. More specifically, the training manager 704 can access, identify, generate, create, and/or determine training input and utilize the training input to train and fine-tune a machine learning model. For instance, the training manager 704 can train the glaucoma diagnosis ML model 410, the glaucoma progression ML model 412, and the RGC count ML model 414, as well as provide evaluation metrics, as discussed above. As discussed, the ML models are trained specifically for a specific diagnosis in some embodiments. For example, an ML model is trained (or the existing ML model is retrained) to diagnose a subject with glaucoma, and another ML model is trained to diagnose a glaucoma progression in the subject.

As illustrated in FIG. 4, the glaucoma diagnosis system 400 also includes a data preprocessor 405 (e.g., “the data preprocessor 110” described above). As discussed, the data preprocessor 405 implements data preprocessing techniques to generate a training dataset for training machine learning models by the training manager 704. The data preprocessor 405 can generate a plurality of relevant features from the input ERG signals 418 to create training datasets for the glaucoma diagnosis ML model 410, the glaucoma progression ML model 412, and the RGC count ML model 414, respectively. The resulting training datasets are provided to the training manager 704 for training, as discussed.

As illustrated in FIG. 4, the glaucoma diagnosis system 400 also includes a machine learning component 406. The machine learning component 406 may host a plurality of machine learning models or other machine learning models, such as the glaucoma diagnosis ML model 410, the glaucoma progression ML model 412, and the RGC count ML model 414. The machine learning component 406 may include an execution environment, libraries, and/or any other data needed to execute the machine learning models. In some embodiments, the machine learning component 406 may be associated with dedicated software and/or hardware resources to execute the machine learning models. As discussed, glaucoma diagnosis ML model 410, the glaucoma progression ML model 412, and the RGC count ML model 414 can be implemented as a decision tree model, a discriminant model, a support vector machine, a nearest neighbor algorithm, or an ensemble classifier or combination of these or other types of ML models as well MLPs, CNNs or combinations of these or other types of neural networks.

Although depicted in FIG. 4 as being hosted by a machine learning component 406, in various embodiments, the ML models may be hosted in multiple machine learning components and/or as part of different components. For example, a glaucoma diagnosis manager can host the glaucoma diagnosis ML model 410. Similarly, a glaucoma progression manager can host the glaucoma progression ML model 412 and the RGC count ML model 414. In various embodiments, the glaucoma diagnosis manager and the glaucoma progression manager can each include their own machine learning component, or other host environments, in which the respective ML models execute.

As illustrated in FIG. 4, the glaucoma diagnosis system 400 also includes the storage manager 408. The storage manager 708 maintains data for the glaucoma diagnosis system 400. The storage manager 408 can maintain data of any type, size, or kind as necessary to perform the functions of the glaucoma diagnosis system 400. The storage manager 408, as shown in FIG. 4, includes the input ERG signals 418. The input ERG signals 418 can include a plurality of ERG signals associated with various subjects, as discussed in additional detail above. In particular, in one or more embodiments, the input ERG signals 418 include ERG signals utilized by the training manager 404 to train the plurality of ML models to generate glaucoma diagnosis predictions 420, glaucoma progression predictions 422, and RGC count predictions 424.

Each of the components 402-408 of the glaucoma diagnosis system 400 and their corresponding elements (as shown in FIG. 4) may be in communication with one another using any suitable communication technologies. It will be recognized that although components 402-408 and their corresponding elements are shown to be separate in FIG. 4, any of components 402-408 and their corresponding elements may be combined into fewer components, such as into a single facility or module, divided into more components, or configured into different components as may serve a particular embodiment.

The components 402-408 and their corresponding elements can comprise software, hardware, or both. For example, components 402-408 and their corresponding elements can comprise one or more instructions stored on a computer-readable storage medium and executable by processors of one or more computing devices. When executed by the one or more processors, the computer-executable instructions of the glaucoma diagnosis system 400 can cause a client device and/or a server device to perform the methods described herein. Alternatively, the components 402-408 and their corresponding elements can comprise hardware, such as a special purpose processing device to perform a certain function or group of functions. Additionally, the components 402-408 and their corresponding elements can comprise a combination of computer-executable instructions and hardware.

Furthermore, the components 402-408 of the glaucoma diagnosis system 400 may, for example, be implemented as one or more stand-alone applications, as one or more modules of an application, as one or more plug-ins, as one or more library functions or functions that may be called by other applications, and/or as a cloud-computing model. Thus, the components 402-408 of the glaucoma diagnosis system 400 may be implemented as a stand-alone application, such as a desktop or mobile application. Furthermore, the components 402-408 of the glaucoma diagnosis system 400 may be implemented as one or more web-based applications hosted on a remote server.

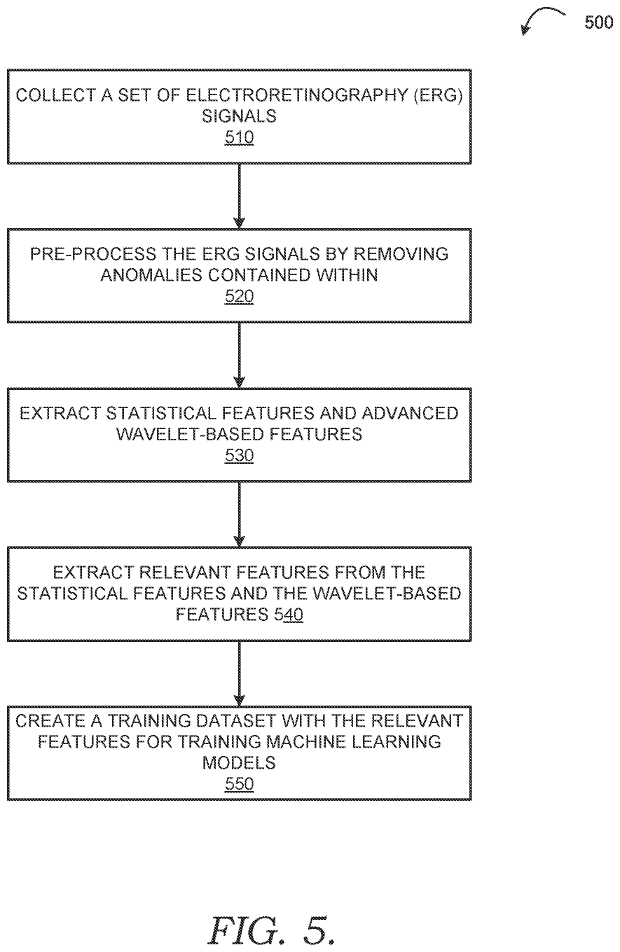

FIGS. 1-4, the corresponding text, and the examples, provide a number of different systems and devices that enable glaucoma diagnosis and glaucoma progression using relevant features extracted from ERG signals, allowing for early-stage detection of glaucoma in subjects. In addition to the foregoing, embodiments can also be described in terms of flowcharts comprising acts and steps to accomplish a particular result. For example, FIGS. 5, 6, and 7 illustrate flowcharts of exemplary methods in accordance with one or more embodiments. The methods described in relation to FIGS. 5, 6, and 7 may be performed with fewer or more steps/acts, or the steps/acts may be performed in differing orders. Additionally, the steps/acts described herein may be repeated or performed in parallel with one another or in parallel with different instances of the same or similar steps/acts.

### Example Flow Diagrams

With reference now to FIGS. 5-7, flow diagrams are provided illustrating various methods. Each block of the methods 500-700 and any other methods described herein comprise a computing process performed using any combination of hardware, firmware, and/or software. For instance, in some embodiments, various functions are carried out by a processor executing instructions stored in memory. In some cases, the methods are embodied as computer-usable instructions stored on computer storage media. In some implementations, the methods are provided by a standalone application, a service or hosted service (standalone or in combination with another hosted service), or a plug-in to another product, to name a few.

FIG. 5 illustrates a flowchart 500 of a series of acts in a method of extracting relevant features from a set of ERG signals to create a training dataset to train machine learning models in early-stage glaucoma diagnosis, in accordance with embodiments of the present disclosure. In one or more embodiments, the method 500 is performed in a digital medium environment that includes the glaucoma diagnosis system 400. The method 500 is intended to be illustrative of one or more methods in accordance with the present disclosure and is not intended to limit potential embodiments. Alternative embodiments can include additional, fewer, or different steps that those articulated in FIG. 5.

As illustrated in FIG. 5, the method 500 includes a step 510 of collecting a set of ERG signals. As discussed, the ERG signals can include ERG signal data that measure the electrical responses of cells in the retinas from animals and/or humans. The ERG signals include OP, STR, rods, rods and cones, Hi rods and cones, cones, Hi cones, flicker, and Hi flicker measurements of retinas.

The method 500 also includes pre-processing, the ERG signals by removing anomalies contained within data of the ERG signals by a data preprocessor. This is illustrated at step 520. Pre-processing includes baseline adjustment, feature extraction, handling missing data, handling outliers, feature scaling, and feature selection on the input ERG signals. In some implementations, the data preprocessor performs baseline adjustments on the input ERG signals as part of the preprocessing process. An ERG signal's baseline (start time) can be different for different animals and testing protocols. Therefore, during baseline adjustment, the measurements can be brought to a common baseline (start time offset to zero) during baseline adjustment. In some embodiments, the data preprocessor performs baseline adjustment techniques such as median filter, linear phase high pass filter, and mean median filter to perform baseline adjustments on the input ERG signals.

The method 500 further includes extracting statistical features and advanced wavelet-based features from the ERG signals using a feature extractor. This is illustrated at step 530. Statistical feature extraction 121 techniques produce statistical features capable of describing the general behavior of ERG signals extracted from the input ERG signals 102. These features include, but are not limited to, measures of central tendency, spread, shape, peaks, derivatives, and correlation.

The feature extractor can implement a variety of techniques to extract the advanced wavelet-based features. These techniques include, but are not limited to, an AR coefficients process, wavelet-based Shannon entropy, multifractal leader estimates, and multiscale wavelet variance estimate. As discussed, Shannon entropy, or information entropy, measures how much information there is in an event. In general, the more certain or deterministic the event is, the less information it will contain. Wavelet leaders are time/space-localized suprema of the discrete wavelet coefficients' absolute value. These suprema are used to calculate the Holder exponents, which characterize the local regularity. Wavelet variance quantifies the degree of variability in a signal by scale, or more precisely, the degree of variability in a signal between octave-band frequency intervals.

The method 500 also includes extracting relevant features from the relevant features (e.g., the statistical and wavelet-based features) using feature selection and extraction techniques. This is illustrated at step 540. The feature selection and extraction techniques include, but are not limited to mRmR Relief, CMIM Correlation Coefficient, Bw-Ratio, Interact, GA, SVM-REF, PCA, Non-Linear PCA, Independent Component Analysis, and correlation based feature selection. Using the extracted relevant features, a training dataset is compiled and created for training machine learning models in glaucoma diagnosis. This is illustrated at step 550.

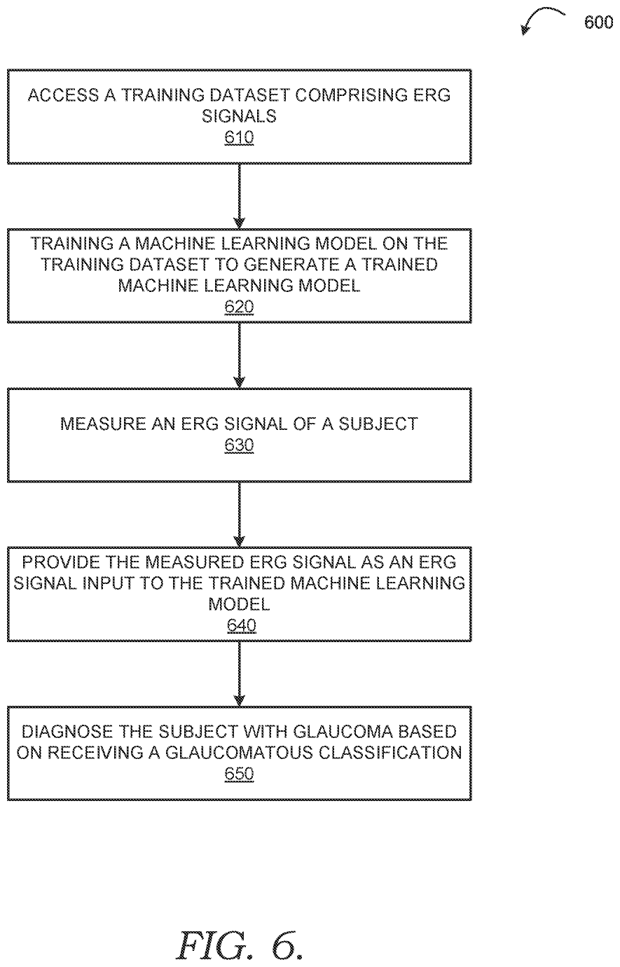

FIG. 6 illustrates a flowchart 600 of a series of acts in a method of training a machine learning model to provide glaucoma diagnosis classifications, in accordance with embodiments of the present disclosure. In one or more embodiments, the method 600 is performed in a digital medium environment that includes the glaucoma diagnosis system 400. The method 600 is intended to be illustrative of one or more methods in accordance with the present disclosure and is not intended to limit potential embodiments. Alternative embodiments can include additional, fewer, or different steps that those articulated in FIG. 6.

As illustrated in FIG. 6, the method 600 includes a step 610 of accessing a training dataset comprising ERG signals. As discussed, the ERG signals can include ERG signal data that measure the electrical responses of cells in the retinas of animals and/or humans. The ERG signals include OP, STR, rods, rods and cones, Hi rods and cones, cones, Hi cones, flicker, and Hi flicker measurements of retinas. Additionally, each ERG signal is associated with a binary label indicating whether the ERG signal is glaucomatous or non-glaucomatous.

The method 600 further includes training a machine learning model on the training dataset. This is illustrated at step 620. A training manager of the glaucoma diagnosis system can teach, guide, tune, and/or train the machine learning model. In particular, the training manager can train the machine learning model based on the training dataset (e.g., input ERG signals). More specifically, the training manager can access, identify, generate, create, and/or determine training input and utilize the training input to train and fine-tune the machine learning model. In some embodiments, the training manager trains a glaucoma diagnosis ML model, a glaucoma progression ML model, and a RGC count ML model, as well as provide evaluation metrics, as discussed above. As discussed, in some embodiments, the ML models are trained specifically for a specific diagnosis. For example, the machine learning model is trained (or the existing ML model retrained) to diagnose a subject with glaucoma, and another ML model is trained to diagnose a glaucoma progression in the subject.

In some embodiments, the training manager evaluates the binary classification predictions using performance evaluation metrics and retrains the machine learning model until a stopping criterion is reached. The training manager can also evaluate the multiclass classification predictions using performance evaluation metrics and retrain the glaucoma progression ML model until a stopping criterion is reached. The training manager can also evaluate the ERG count predictions using performance evaluation metrics and retrain the RGC count ML model until a stopping criterion is reached.

The method 600 also includes measuring an ERG signal of a subject. This is illustrated at step 630. Using known techniques, equipment, and devices, a measurement of the ERG signal is collected. As discussed, the ERG signals can include ERG signal data that measure the electrical responses of cells in the retinas of animals and/or humans. The ERG signals include OP, STR, rods, rods and cones, Hi rods and cones, cones, Hi cones, flicker, and Hi flicker measurements of retinas.

Upon measurement, the ERG signal is provided as an ERG signal input to the trained machine-learning model. This is illustrated at step 640. Once inputted, the machine learning model provides a binary classification of a glaucoma diagnosis (e.g., glaucomatous and non-glaucomatous). If the binary classification returns as glaucomatous, the classification can act as a diagnosis for the subject. This is illustrated at step 650.

In some embodiments, the measured ERG signal is provided as an ERG signal to a glaucoma progression ML model. Once inputted, the glaucoma progression ML model provides a multiclass classification of the progression of glaucoma in the subject. Additionally, or alternatively, the measured ERG signal is provided as an ERG signal to an RGC count ML model. Once inputted, the RGC count ML model provides an RGC count prediction for the subject based on the provided ERG signal.

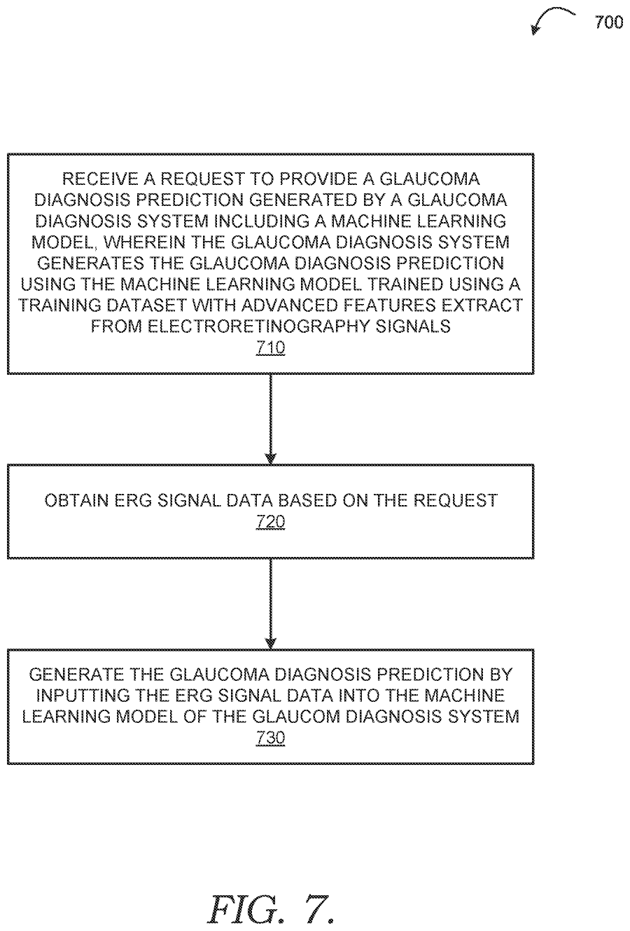

FIG. 7 illustrates a flowchart of a series of acts in a method of glaucoma diagnosis with a machine learning framework using ERG signals in accordance with one or more embodiments. In one or more embodiments, the method 700 is performed in a digital medium environment that includes the glaucoma diagnosis system 400. The method 700 is intended to be illustrative of one or more methods in accordance with the present disclosure and is not intended to limit potential embodiment. Alternative embodiments can include additional, fewer, or different steps than those articulated in FIG. 7.

As illustrated in FIG. 7, the method 700 includes a step 710 of receiving a request to provide a glaucoma diagnosis generated by a glaucoma diagnosis system, including a plurality of machine learning models. The glaucoma diagnosis system generates the glaucoma diagnosis by inputting ERG signal data in a trained machine learning model that provide a binary classification of glaucomatous or non-glaucomatous.

In some embodiments, the glaucoma diagnosis system includes a glaucoma diagnosis ML model to predict a glaucoma diagnosis (e.g., glaucomatous or non-glaucomatous), a glaucoma progression ML model to predict a glaucoma progression based on IOP as normal, high, or glaucomatous, and an RGC count ML model to predict an RGC count based on a regression analysis. In some embodiments, the glaucoma diagnosis system trains the machine learning models using a set of ERG signals. The ERG signals contain data including OP, STR, rods, rods and cones, Hi rods and cones, Cones, Hi cones, flicker, and Hi flicker data.

As illustrated in FIG. 7, the method 700 includes a step 720 of obtaining an ERG signal of a subject. For example, the ERG signal may include information relating to a retina, as discussed above. The glaucoma diagnosis system may be implemented as part of a diagnostics system or as a separate system, making the glaucoma diagnosis available to a diagnostics system for evaluation.

As illustrated in FIG. 7, the method 700 includes a step 730 of generating a glaucoma diagnosis based on the ERG signal and the request. In some embodiments, generating a glaucoma diagnosis further includes generating a glaucoma progression prediction and an RGC count prediction. In some implementations, the glaucoma diagnosis, glaucoma progression, and RGC count predictions are provided in a diagnostics report.

### Example Computing Environment

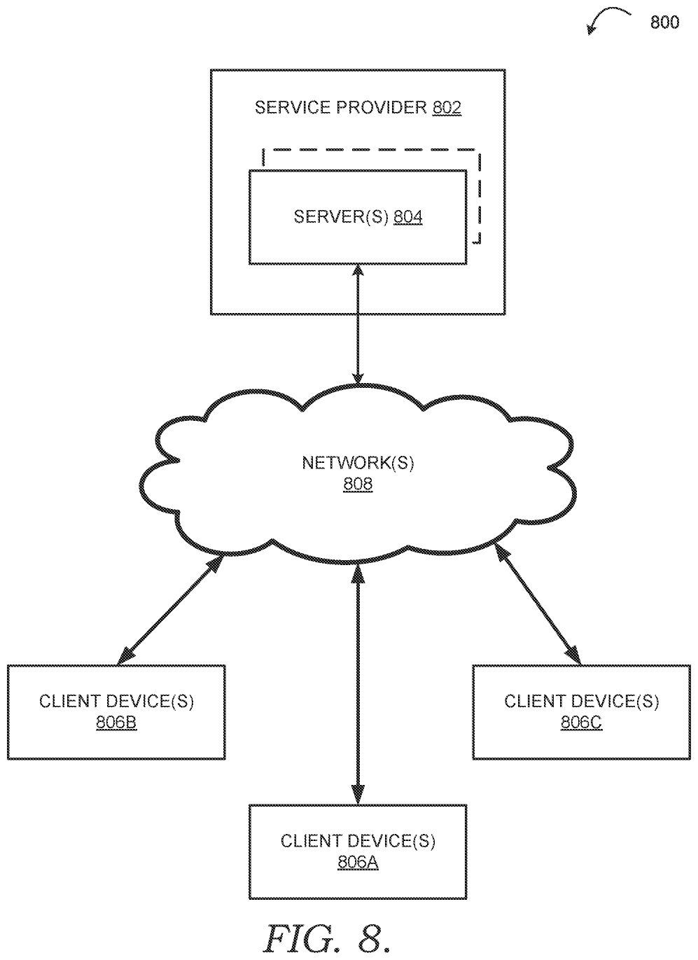

FIG. 8 illustrates a schematic diagram of an exemplary computing environment 800 in which the glaucoma diagnosis system 400 can operate in accordance with one or more embodiments of the present disclosure. In one or more embodiments, the computing environment 800 includes a service provider 802, which may include one or more servers 804 connected to a plurality of client devices 806A-806C via one or more networks 808. The client devices 806A-806C, the one or more networks 808, the service provider 802, and the one or more servers 804 may communicate with each other or other components using any communication platforms and technologies suitable for transporting data and/or communication signals, including any known communication technologies, devices, media, and protocols supportive of remote data communications, examples of which will be described in more detail below with respect to FIG. 9.

Although FIG. 8 illustrates a particular arrangement of the client devices 806A-806C, the one or more networks 808, the service provider 802, and the one or more servers 804, various additional arrangements are possible. For example, the client devices 806A-806C may directly communicate with the one or more servers 804, bypassing the network 808. Or alternatively, the client devices 806A-806C may directly communicate with each other. The service provider 802 may be a public cloud service provider, which owns and operates its own infrastructure in one or more data centers and provides this infrastructure to customers and end users on demand to host applications on the one or more servers 804. The servers may include one or more hardware servers (e.g., hosts), each with its own computing resources (e.g., processors, memory, disk space, networking bandwidth, etc.), which may be securely divided between multiple customers, each of which hosts their own applications on the one or more servers.

In some embodiments, the service provider may be a private cloud provider who maintains cloud infrastructure for a single organization. The one or more servers 804 may similarly include one or more hardware servers, each with its own computing resources, which are divided among applications hosted by the one or more servers for use by members of the organization or their customers.

Similarly, although the computing environment 800 of FIG. 8 is depicted as having various components, the computing environment 800 may have additional or alternative components. For example, the environment 800 can be implemented on a single computing device with the glaucoma diagnosis system 400. In particular, the glaucoma diagnosis system 400 may be implemented in whole or in part on the client device 802A.

As illustrated in FIG. 8, the environment 800 may include client devices 806A-806C. The client devices 806A-806C may comprise any computing device. For example, client devices 806A-806C may comprise one or more personal computers, laptop computers, mobile devices, mobile phones, tablets, special purpose computers, TVs, or other computing devices, including computing devices described below with regard to FIG. 8. Although three client devices are shown in FIG. 8, it will be appreciated that client devices 806A-806C may comprise any number of client devices (greater or smaller than shown).

Moreover, as illustrated in FIG. 8, the client devices 806A-806C and the one or more servers 804 may communicate via one or more networks 808. The one or more networks 808 may represent a single network or a collection of networks (such as the Internet, a corporate Intranet, a virtual private network (VPN), a local area network (LAN), a wireless local network (WLAN), a cellular network, a wide area network (WAN), a metropolitan area network (MAN), or a combination of two or more such networks. Thus, the one or more networks 808 may be any suitable network over which the client devices 806A-806N may access service provider 802 and server 804, or vice versa. The one or more networks 808 will be discussed in more detail below with regard to FIG. 9.

In addition, the environment 800 may also include one or more servers 804. The one or more servers 804 may generate, store, receive, and transmit any type of data, including input ERG signals 418, glaucoma diagnosis prediction 420, glaucoma progression predictions 422, RGC count predictions 424, or other information. For example, a server 804 may receive data from a client device, such as the client device 806A, and send the data to another client device, such as the client device 802B and/or 802C. The server 804 can also transmit electronic messages between one or more users of the environment 800. In one example embodiment, the server 804 is a data server. The server 804 can also comprise a communication server or a web-hosting server. Additional details regarding the server 804 will be discussed below with respect to FIG. 9.

As mentioned, in one or more embodiments, the one or more servers 804 can include or implement at least a portion of the glaucoma diagnosis system 400. In particular, the glaucoma diagnosis system 400 can comprise an application running on the one or more servers 804, or a portion of the glaucoma diagnosis system 400 can be downloaded from the one or more servers 804. For example, the glaucoma diagnosis system 400 can include a web hosting application that allows the client devices 806A-806C to interact with content hosted at the one or more servers 804. To illustrate, in one or more embodiments of the environment 800, one or more client devices 806A-806C can access a webpage supported by the one or more servers 804. In particular, the client device 806A can run a web application (e.g., a web browser) to allow a user to access, view, and/or interact with a webpage or website hosted at the one or more servers 804.

Upon the client device 806A accessing a webpage or other web application hosted at the one or more servers 804, in one or more embodiments, the one or more servers 804 can provide access to one or more digital images (e.g., the input ERG signals 418) stored at the one or more servers 804. Moreover, the client device 806A can receive a request (i.e., via user input) to perform glaucoma diagnosis and provide the request to the one or more servers 804. Upon receiving the request, the one or more servers 804 can automatically perform the methods and processes described above. The one or more servers 804 can provide all or portions of the glaucoma diagnosis predictions to the client device 806A for display to the user. The one or more servers 804 can also host a diagnostics application used to provide diagnostics to a subject.

As just described, the glaucoma diagnosis system 400 may be implemented in whole, or in part, by the individual elements 802-808 of the computing environment 800. It will be appreciated that although certain components of the glaucoma diagnosis system 400 are described in the previous examples with regard to particular elements of the computing environment 800, various alternative implementations are possible. For instance, in one or more embodiments, the glaucoma diagnosis system 400 is implemented on any of the client devices 806A-C. Similarly, in one or more embodiments, the glaucoma diagnosis system 400 may be implemented on the one or more servers 804. Moreover, different components and functions of the glaucoma diagnosis system 400 may be implemented separately among client devices 806A-806C, the one or more servers 804, and the network 808.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links that can be used to carry desired program code means in the form of computer-executable instructions or data structures and which can be accessed by a general-purpose or special-purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data, which, when executed at a processor, cause a general-purpose computer, special-purpose computer, or special-purpose processing device to perform a certain function or group of functions. In some embodiments, computer-executable instructions are executed on a general-purpose computer to turn the general-purpose computer into a special-purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including personal computers, desktop computers, laptop computers, message processors, handheld devices, multiprocessor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud computing model can also be deployed using different deployment models such as private cloud, community cloud, public cloud, hybrid cloud, and so forth. In this description and in the claims, a “cloud-computing environment” is an environment in which cloud computing is employed.

### Example Operating Environment

Having described an overview of embodiments of the present technology, an example operating environment in which embodiments of the present technology may be implemented is described in order to provide a general context for various aspects of the present technology. Referring now to FIG. 9, in particular, an exemplary operating environment for implementing embodiments of the present technology is shown and designated generally as computing device 900. Computing device 900 is but one example of a suitable computing environment and is not intended to suggest any limitation as to the scope of use or functionality of the technology. Neither should computing device 900 be interpreted as having any dependency or requirement relating to any one or combination of components illustrated.

The technology of the present disclosure may be described in the general context of computer code or machine-useable instructions, including computer-executable instructions such as program modules, being executed by a computer or other machines, such as a personal data assistant or other handheld devices. Generally, program modules, including routines, programs, objects, components, data structures, etc., refer to code that performs particular tasks or implement particular abstract data types. The technology may be practiced in a variety of system configurations, including hand-held devices, consumer electronics, general-purpose computers, more specialty computing devices, etc. The technology may also be practiced in distributed computing environments where tasks are performed by remote-processing devices that are linked through a communications network.

With reference to FIG. 9, computing device 900 includes bus 910 that directly or indirectly couples the following devices: memory 912, one or more processors 914, one or more presentation components 916, input/output ports 918, input/output components 920, and illustrative power supply 922. Bus 910 represents what may be one or more buses (such as an address bus, data bus, or combination thereof). Although the various blocks of FIG. 9 are shown with lines for the sake of clarity, in reality, delineating various components is not so clear, and metaphorically, the lines would more accurately be grey and fuzzy. For example, one may consider a presentation component, such as a display device, or an I/O component. In addition, processors have memory. We recognize that such is the nature of the art and reiterate that the diagram of FIG. 9 merely illustrates an example computing device that can be used in connection with one or more embodiments of the present technology. A distinction is not made between such categories as “workstation,” “server,” “laptop,” “hand-held device,” etc., as all are contemplated within the scope of FIG. 9 and reference to “computing device.”

Computing device 900 typically includes a variety of computer-readable media. Computer-readable media can be any available media that can be accessed by computing device 900 and includes both volatile and nonvolatile media, removable and non-removable media. By way of example, and not limitation, computer-readable media may comprise computer storage media and communication media.

Computer storage media include volatile and nonvolatile, removable and non-removable media implemented in any method or technology for storage of information such as computer-readable instructions, data structures, program modules, or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing device 800. Computer storage media excludes signals per se.

Communication media typically embodies computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared, and other wireless media. Combinations of any of the above should also be included within the scope of computer-readable media.

Memory 912 includes computer storage media in the form of volatile or nonvolatile memory. The memory may be removable, non-removable, or a combination thereof. Examples of hardware devices include solid-state memory, hard drives, optical-disc drives, etc. Computing device 900 includes one or more processors that read data from various entities, such as memory 912 or I/O components 920. Presentation component(s) 916 presents data indications to a user or other device. Examples of presentation components include a display device, speaker, printing component, vibrating component, etc.

I/O ports 918 allow computing device 900 to be logically coupled to other devices, including I/O components 920, some of which may be built in. Illustrative components include a microphone, joystick, game pad, satellite dish, scanner, printer, wireless device, etc.

Having identified various components in the present disclosure, it should be understood that any number of components and arrangements may be employed to achieve the desired functionality within the scope of the present disclosure. For example, the components in the embodiments depicted in the figures are shown with lines for the sake of conceptual clarity. Other arrangements of these and other components may also be implemented. For example, although some components are depicted as single components, many ofthe elements described herein may be implemented as discrete or distributed components or in conjunction with other components, and in any suitable combination and location. Some elements may be omitted altogether. Moreover, various functions described herein as being performed by one or more entities may be carried out by hardware, firmware, and/or software, as described below. For instance, various functions may be carried out by a processor executing instructions stored in memory. As such, other arrangements and elements (e.g., machines, interfaces, functions, orders, and groupings of functions, etc.) can be used in addition to or instead of those shown.

The subject matter of the present invention is described with specificity herein to meet statutory requirements. However, the description itself is not intended to limit the scope of this patent. Rather, the inventor has contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described. For purposes of this disclosure, words such as “a” and “an,” unless otherwise indicated to the contrary, include the plural as well as the singular. Thus, for example, the requirement of “a feature” is satisfied where one or more features are present.

The present invention has been described in relation to particular embodiments, which are intended in all respects to be illustrative rather than restrictive. Alternative embodiments will become apparent to those of ordinary skill in the art to which the present invention pertains without departing from its scope.

From the foregoing, it will be seen that this invention is one well adapted to attain all the ends and objects set forth above, together with other advantages, which are obvious and inherent to the system and method. It will be understood that certain features and subcombinations are of utility and may be employed without reference to other features and subcombinations. This is contemplated by and is within the scope of the claims.

The following embodiments represent exemplary embodiments of concepts contemplated herein. Any one of the following embodiments may be combined in a multiple dependent manner to depend from one or more other clauses. Further, any combination of dependent embodiments (e.g., clauses that explicitly depend from a previous clause) may be combined while staying within the scope of aspects contemplated herein. The following clauses are exemplary in nature and are not limiting.

Clause 1. A method comprising: collecting a set of electroretinography (ERG) signals having measurement information relating to electrical responses of cells in retinas; pre-processing the ERG signals by removing anomalies contained within data of the ERG signals; extracting statistical features and wavelet-based features from the pre-processed ERG signals; extracting features from the statistical features and the wavelet-based features based at least in part on using feature selection and extraction techniques; and generating a training dataset with the features for training at least one machine learning model in glaucoma diagnosis predictions. Thus, the illustrative embodiment provides technological improvements over conventional techniques by implementing a glaucoma diagnosis system that extracts more efficient and relevant features (e.g., advanced statistical features and advanced wavelet-based features) for providing glaucoma diagnosis, glaucoma progression, and retinal ganglion cell count predictions.

Clause 2. The method of claim 1, further comprising: training the machine learning model to produce binary classification predictions of glaucoma using the training dataset; evaluating the binary classification predictions using performance evaluation metrics; and retraining the machine learning model until a stopping criterion is reached.

Clause 3. The method of claim 1 or 2, further comprising: training the machine learning model to produce multiclass classification predictions relating to stages of glaucoma progression using the training dataset; evaluating the multiclass classification predictions using performance evaluation metrics; and retraining the machine learning model until a stopping criterion is reached.

Clause 4. The method of claim 1, or 2 or 3, further comprising: training the machine learning model to produce retinal ganglion cell (RGC) count predictions to provide quantitative assessments of visual functions using the training dataset; evaluating the ERG count predictions using performance evaluation metrics; and retraining the machine learning model until a stopping criterion is reached.

Clause 5. The method of claim 1, or 2, or 3, or 4, wherein the machine learning model is an artificial neural network.

Clause 6. The method of claim 1, or 2, or 3, or 4, or 5, wherein the wavelet-based features include coefficients from an autoregressive model that describe power-law behaviors at various resolutions and wavelet variance of the ERG signals.

Clause 7. The method of claim 1, or 2, or 3, or 4, or 5, or 6, wherein the wavelet-based features include Shannon entropy values for maximal overlap discrete wavelet packet transform (MOD-PWT).

Clause 8. The method of claim 1, or 2, or 3, or 4, or 5, or 6, or 7, wherein the wavelet-based features include multifractal wavelet leader estimates of a second cumulant of scaling exponents and a range of Holder exponents.

Clause 9. A system for detecting glaucoma, the system comprising: at least one processor; and one or more computer storage media storing computer executable instructions that when executed by the at least one processor, cause the at least one processor to perform operations comprising: accessing a training data set comprising electroretinography (ERG) signals, each of the ERG signals being associated with a binary label selected from glaucomatous and non-glaucomatous; training a machine learning model on the training data set to generate a trained machine learning model; obtaining ERG signal data relating to a subject; providing the ERG signal data as an ERG signal input to the trained machine learning model; and receiving a classification from the machine learning model determined in response to the ERG signal input and relating to the subject. Thus, the illustrative embodiment provides technological improvements over conventional techniques by implementing a glaucoma diagnosis system that trains machine learning models using more efficient and relevant features (e.g., advanced statistical features and advanced wavelet-based features) that provide glaucoma diagnosis, glaucoma progression, and retinal ganglion cell count predictions.

Clause 10. The system of claim 9, further comprising reducing dimensionality of the ERG signals of the training data set by extracting ERG signal features.

Clause 11. The system of claim 9, or 10, wherein the machine learning model is a decision tree model, a discriminant model, a support vector machine, a nearest neighbor algorithm, or an ensemble classifier.

Clause 12. The system of claim 9, or 10, or 11, further comprising treating the subject for glaucoma based on the glaucomatous classification.

Clause 13. The system of claim 9, or 10, or 11, or 12, wherein training the machine learning model comprises: training the machine learning model to produce binary classification predictions of glaucoma using the training dataset; evaluating the binary classification predictions using performance evaluation metrics; and retraining the machine learning model until a stopping criterion is reached.

Clause 14. The system of claim 9, or 10, or 11, or 12, or 13, wherein training the machine learning model comprises: training the machine learning model to produce multiclass classification predictions relating to stages of glaucoma progression using the training dataset; evaluating the multiclass classification predictions using performance evaluation metrics; and retraining the machine learning model until a stopping criterion is reached.

Clause 15. The system of claim 9, or 10, or 11, or 12, or 13, or 14, wherein training the machine learning model comprises: training the machine learning model to produce retinal ganglion cell (ERG) count predictions to provide quantitative assessments of visual functions using the training dataset; evaluating the ERG count predictions using performance evaluation metrics; and retraining the machine learning model until a stopping criterion is reached.

Clause 16. The system of claim 9, or 10, or 11, or 12, or 13, or 14, or 15, wherein the training dataset include extracted statistical features and wavelet-based features based at least in part on using feature selection and extraction techniques on the ERG signals.

Clause 17. A non-transitory computer readable storage medium including instructions stored thereon which, when executed by a processor, cause the processor to: receive a request to provide a glaucoma diagnosis prediction generated by a glaucoma diagnosis system including a machine learning model, wherein the glaucoma diagnosis system generates the glaucoma diagnosis prediction using the machine learning model trained using a training dataset with advanced features extracted from electroretinography (“ERG”) signals; obtain ERG signal data based on the request; and generate the glaucoma diagnosis prediction based at least in part on inputting the ERG signal data into the machine learning model of the glaucoma diagnosis framework. Thus, the illustrative embodiment provides technological improvements over conventional techniques by implementing a glaucoma diagnosis system that provides machine learning models trained using more efficient and relevant features (e.g., advanced statistical features and advanced wavelet-based features) so that they provide glaucoma diagnosis, glaucoma progression, and retinal ganglion cell count predictions.

Clause 18. The non-transitory computer-readable storage medium of claim 17, further comprising instructions to cause the processor to: receive a second request to provide a glaucoma progression prediction generated by the glaucoma diagnosis system including a second machine learning model, wherein the glaucoma diagnosis system generates the glaucoma progression prediction using the second machine learning model trained using the training dataset; and generate the glaucoma progression prediction by inputting the ERG signal data into the second machine learning model of the glaucoma diagnosis framework.

Claim 19. The non-transitory computer-readable storage medium of claim 17, or 18, further comprising instructions to cause the processor to: receive a second request to provide a retinal ganglion cell (RGC) count prediction generated by the glaucoma diagnosis system including a second machine learning model, wherein the glaucoma diagnosis system generates the RGC count prediction using the second machine learning model trained using the training dataset; and generate the RGC count prediction by inputting the ERG signal data into the second machine learning model of the glaucoma diagnosis framework.

Clause 20. The non-transitory computer-readable storage medium of claim 17, or 18, or 19, wherein the advanced features include extracted statistical features and wavelet-based features based at least in part on using feature selection and extraction techniques on the ERG signals.

What is claimed is:

1. A method comprising: collecting a set of electroretinography (ERG) signals having measurement information relating to electrical responses of cells in retinas; pre-processing the ERG signals by removing anomalies contained within data of the ERG signals; extracting statistical features and wavelet-based features from the pre-processed ERG signals; extracting features from the statistical features and the wavelet-based features based at least in part on using feature selection and extraction techniques; and generating a training dataset with the features for training at least one machine learning model in glaucoma diagnosis predictions.

2. The method of claim 1, further comprising: training the machine learning model to produce binary classification predictions of glaucoma using the training dataset; evaluating the binary classification predictions using performance evaluation metrics; and retraining the machine learning model until a stopping criterion is reached.

3. The method of claim 1, further comprising: training the machine learning model to produce multiclass classification predictions relating to stages of glaucoma progression using the training dataset; evaluating the multiclass classification predictions using performance evaluation metrics; and retraining the machine learning model until a stopping criterion is reached.

4. The method of claim 1, further comprising: training the machine learning model to produce retinal ganglion cell (RGC) count predictions to provide quantitative assessments of visual functions using the training dataset; evaluating the ERG count predictions using performance evaluation metrics; and retraining the machine learning model until a stopping criterion is reached.

5. The method of claim 4, wherein the machine learning model is an artificial neural network.

6. The method of claim 1, wherein the wavelet-based features include coefficients from an autoregressive model that describe power-law behaviors at various resolutions and wavelet variance of the ERG signals.

7. The method of claim 1, wherein the wavelet-based features include Shannon entropy values for maximal overlap discrete wavelet packet transform (MOD-PWT).

8. The method of claim 1, wherein the wavelet-based features include multifractal wavelet leader estimates of a second cumulant of scaling exponents and a range of Holder exponents.

9. A system for detecting glaucoma, the system comprising: at least one processor; and one or more computer storage media storing computer executable instructions that when executed by the at least one processor, cause the at least one processor to perform operations comprising: accessing a training data set comprising electroretinography (ERG) signals, each of the ERG signals being associated with a binary label selected from glaucomatous and non-glaucomatous; training a machine learning model on the training data set to generate a trained machine learning model; obtaining ERG signal data relating to a subject; providing the ERG signal data as an ERG signal input to the trained machine learning model; and receiving a classification from the machine learning model determined in response to the ERG signal input and relating to the subject.

10. The system of claim 9, further comprising reducing dimensionality of the ERG signals of the training data set by extracting ERG signal features.

11. The system of claim 9, wherein the machine learning model is a decision tree model, a discriminant model, a support vector machine, a nearest neighbor algorithm, or an ensemble classifier.

12. The system of claim 9, further comprising treating the subject for glaucoma based on the glaucomatous classification.

13. The system of claim 9, wherein training the machine learning model comprises: training the machine learning model to produce binary classification predictions of glaucoma using the training dataset; evaluating the binary classification predictions using performance evaluation metrics; and retraining the machine learning model until a stopping criterion is reached.

14. The system of claim 9, wherein training the machine learning model comprises: training the machine learning model to produce multiclass classification predictions relating to stages of glaucoma progression using the training dataset; evaluating the multiclass classification predictions using performance evaluation metrics; and retraining the machine learning model until a stopping criterion is reached.

15. The system of claim 9, wherein training the machine learning model comprises: training the machine learning model to produce retinal ganglion cell (ERG) count predictions to provide quantitative assessments of visual functions using the training dataset; evaluating the ERG count predictions using performance evaluation metrics; and retraining the machine learning model until a stopping criterion is reached.

16. The system of claim 9, wherein the training dataset include extracted statistical features and wavelet-based features based at least in part on using feature selection and extraction techniques on the ERG signals.

17. A non-transitory computer readable storage medium including instructions stored thereon which, when executed by a processor, cause the processor to: receive a request to provide a glaucoma diagnosis prediction generated by a glaucoma diagnosis system including a machine learning model, wherein the glaucoma diagnosis system generates the glaucoma diagnosis prediction using the machine learning model trained using a training dataset with advanced features extracted from electroretinography (“ERG”) signals; obtain ERG signal data based on the request; and generate the glaucoma diagnosis prediction based at least in part on inputting the ERG signal data into the machine learning model of the glaucoma diagnosis framework.

18. The non-transitory computer-readable storage medium of claim 17, further comprising instructions to cause the processor to: receive a second request to provide a glaucoma progression prediction generated by the glaucoma diagnosis system including a second machine learning model, wherein the glaucoma diagnosis system generates the glaucoma progression prediction using the second machine learning model trained using the training dataset; and generate the glaucoma progression prediction by inputting the ERG signal data into the second machine learning model of the glaucoma diagnosis framework.

19. The non-transitory computer-readable storage medium of claim 17, further comprising instructions to cause the processor to: receive a second request to provide a retinal ganglion cell (RGC) count prediction generated by the glaucoma diagnosis system including a second machine learning model, wherein the glaucoma diagnosis system generates the RGC count prediction using the second machine learning model trained using the training dataset; and generate the RGC count prediction by inputting the ERG signal data into the second machine learning model of the glaucoma diagnosis framework.

20. The non-transitory computer-readable storage medium of claim 17, wherein the advanced features include extracted statistical features and wavelet-based features based at least in part on using feature selection and extraction techniques on the ERG signals.
